(19) United States
(12) Patent Application Publication
(10) Pub. No.: US 2007/0255056 A1
(43) Pub. Date: Nov. 1, 2007

(54) Method for manufacture of compounds related to the class of substituted sulfonyl urea anti-diabetics

(76) Inventors: Ashok Kumar, Mumbai (IN); Satish Rajanikant Soudagar, Mumbai (IN); Pramil Kumar Mathur, Maharashtra (IN); Arpana Mathur, Maharashtra (IN); Dadaso Shankar Dalavi, Maharashtra (IN); Sanjay Tukaram Gunjal, Mumbai (IN); Uttamrao Arjunrao Sawant, Thane (IN); Ashvini Saxena, Ratlam (IN); Bimal Kumar Srivastava, Ratlam (IN); Praveen Kumar Singh, Ratlam (IN); Jagdish Sankla, Ratlam (IN); Vijay Dhurandhare, Ratlam (IN)

Correspondence Address: BLANK ROME LLP, 600 NEW HAMPSHIRE AVENUE, N.W., WASHINGTON, DC, 20037, US

(21) Appl. No.: 11/371,911

(22) Filed: Mar. 10, 2006

(30) Foreign Application Priority Data
Mar. 11, 2005 (IN) 271/MUM/2005
Dec. 27, 2005 (IN) 1627/MUM/2005

(51) Int. Cl. C07C 311/21; C07D 207/24; C07D 209/52; C07D 223/04; C07D 295/14; C07D 413/12

(52) U.S. Cl. 540/603; 540/606; 544/390; 548/248; 548/515; 548/538; 564/86; 564/93

(57) ABSTRACT

The present invention relates to a process for preparation of sulfonyl urea compounds in high conversion rates and purity. More specifically, this invention relates to a process for manufacture of sulfonyl urea class of anti-diabetic pharmaceutical drugs in higher purity and yield. The process may effectively and economically be used to produce anti-diabetic drugs, such as glimepiride, glipizide, gliclazide, glibenclamide, glibornuride, and glisoxepide.

This application claims priority from Indian Patent Application Nos. 271/MUM/2005, filed Mar. 11, 2005, and 1627/MUM/2005, filed Dec. 27, 2005, both of which are incorporated herein by reference.

### FIELD OF INVENTION

The present invention relates to a process for preparation of sulfonyl urea compounds in high conversion rates and purity. More specifically, this invention relates to a process for manufacture of a sulfonyl urea class of anti-diabetic pharmaceutical drugs in higher purity and yield.

### BACKGROUND OF THE INVENTION

Sulfonyl urea compounds are a class of anti-diabetic drugs which are characterized by the general structure of Formula I, 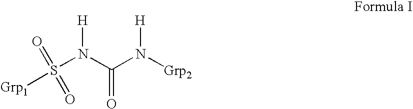
(where Grp1 and Grp2 are defined below) which, as the substance or in the form of their physiologically tolerated salts, are known to have hypoglycemic properties distinguished by a powerful lowering of blood glucose level, and thus, find use in the treatment of diabetics.

Compounds such as Glimepiride is one of the new generation sulfonyl urea anti-diabetic molecules and is one of the best sulfonyl urea drugs in the clinical appraisal. It has been approved by the FDA to treat diabetes type II, which cannot be effectively treated by the dietary adjustment or exercise. In general, these compounds lower blood glucose in non-insulin dependent diabetic mellitus (Type-II diabetic mellitus), by stimulating the release of insulin from functioning pancreatic beta cells. It may also be used along with insulin. Therefore, the synthesis of these anti-diabetic sulfonyl urea drugs in high purity is of extreme importance and highly desirable.

The pharmaceutically valuable drugs in this class include drugs such a glimepiride (Formula V), glipizide (Formula VII), gliclazide (Formula VIII), glibenclamide (Formula III), glibornuride (Formula VI), glisoxepide (Formula II) which are marketed for use in the therapy of type I or II diabetes mellitus. The structures of these compounds are given below: 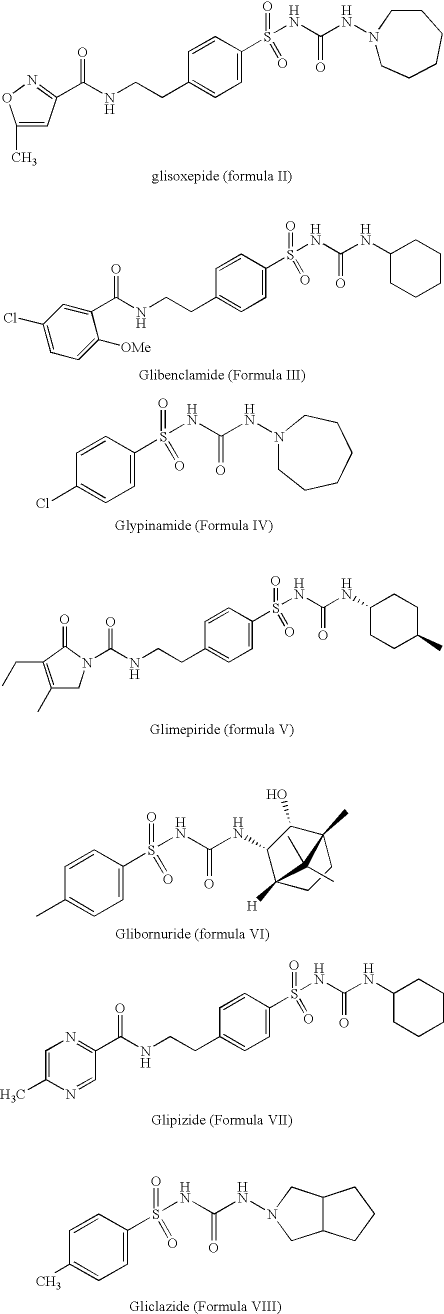
A common structural feature in many of these anti-diabetic drugs is the presence of a sulfonyl urea moiety. Various known processes provide the sulfonyl urea moiety on the compound, many of which involve any one of the approaches listed below:
a) coupling of an isocyanate intermediate with sulphonamide in presence of an inorganic base according to following scheme
   R¹SO2NH2+R²NCO→R¹SO2NHCONH—R²;
b) condensation of a carbamate of sulphonamide intermediate with an amino-intermediate according to following scheme
   R¹SO2NHCOOR³+R²NH2→R¹SO2NHCONH—R²; or
c) Condensation of a sulphonamide compound with an activated carbamate according to the following reaction scheme
   R¹SO2NH2+R²NH—COO—R⁴→R¹SO2NHCONH—R²

In these known processes, the resultant sulfonyl urea moiety formation was found to be incomplete resulting in poor yield and quality of these hypoglycemic pharmaceuticals, because the unreacted sulphonamide intermediates are found to contaminate the final product. Moreover, the use of isocyanate poses considerable process hazards, as well as handling harzards on industrial scale. Additionally, isocyanate generates polymeric impurities during reaction, as well as during storage, due to polymerization/heat instability of the reagent under the reaction conditions, resulting in tedious isolation/purification. Another drawback of these known processes is the presence of impurities of corresponding sulphonamides, either due to incomplete reaction or due to hydrolysis of the product under the reported conditions.

In the process method c), it is observed that for transformation into sulfonyl urea, a carbamate with R4 having a (−)-I group seems to work; however, the reaction is no longer complete. There are reports (U.S. Pat. Nos. 3,787,491 and 3,770,761) attempting the process of method (b) in presence of bases, such as pyridine. It has been observed that the presence of a base does not ameliorate the above mentioned drawbacks and added no observable advantage.

Therefore, there remains a need for a synthetic process for producing sulfonyl urea compounds, especially pharmaceutically useful anti-diabetic drugs, having fast reaction rates with complete conversion, and effective purification methods and improved output.

### SUMMARY OF THE INVENTION

The object of this invention is to develop a rugged synthetic process for sulfonyl urea compounds, especially pharmaceutically useful anti-diabetic drugs where purity is of utmost importance, that consists of fast reaction rate with complete conversion, effective purification method, and improved output. Moreover, the present invention deals with improvements over the synthetic pathway disclosed in the prior art, which ameliorates most of the problems, associated with purity and yield.

Accordingly, the present invention relates to a process for providing high purity sulfonyl urea compounds in high throughput conditions. The sulfonyl urea compounds are preferably the pharmaceutically useful anti-diabetic drugs, such as glimepride, gliclazide, glipizide, Glibenclamide, Glibornuride, Glisoxepide, etc, where the purity and economy of the compound are of utmost importance.

In one aspect of the present invention, there is provided a process for preparation of sulfonyl urea compounds of Formula I, 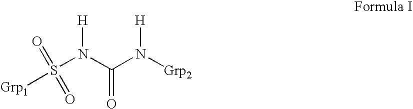
where Grp1 is any appropriately substituted or unsubstituted aryl residue, or a substituent group selected from residues A, B, C, D, E, or F given below: 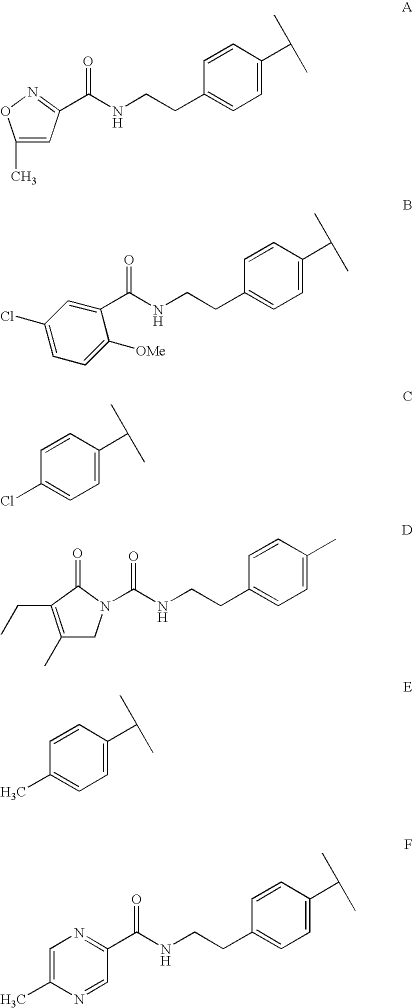
wherein, Grp2 is any unsubstituted or substituted cycloalkyl, polycyclic ring or heterocyclic ring, or a substituent selected from the residues P, Q, R, S, T or similar inert groups

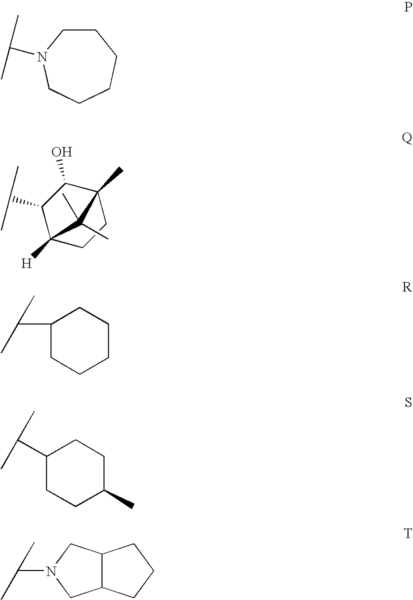

The process comprises reacting a carbamate of Formula IX (wherein Grp1 is as previously defined; and R′ is an alkyl or aryl residue) with an amine compound of Formula X (wherein Grp2 is as previously defined) 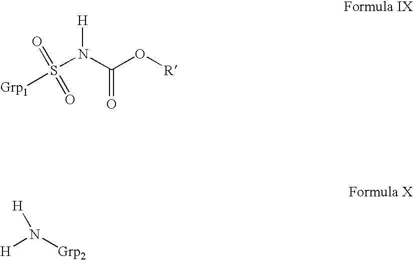
The reaction preferably takes place in presence of an activation catalyst and in a solvent medium to form the sulfonyl urea compound of Formula I. Preferably, the activation catalyst is 4-pyrrolidinopyridine, 4-(dimethylamino)pyridine (DMAP), or the like, with DMAP being the most preferred catalyst.

Preferably, the solvent is a high boiling aromatic hydrocarbon solvent, such as toluene. The reaction is preferably carried out at reflux temperature with optional removal of by-product (alcohol) from the reaction medium by distillation or other means, such as Dean Stark arrangement.

In another aspect of the present invention, there is provided a process for the manufacture of sulfonyl urea compounds of Formula I in higher conversion rates, which comprises reacting a carbamate of Formula XII (wherein Grp2 is as defined above; and R′ is an alkyl or aryl residue) with a sulphonamide of Formula XI (wherein the Grp1 is as define above) 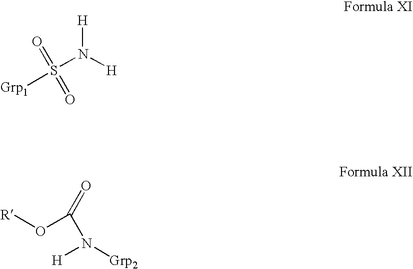
The reaction preferably takes place in the presence of a base and an activation catalyst which is preferably selected from 4-pyrrolidinopyridine, 4-(dimethylamino)pyridine (DMAP), and the like. The base may be any organic or inorganic base. Furthermore, the catalyst of the present invention may be used in excess to also serve as the base; e.g. DMAP, when used in excess, may also serve as an additional base. In such case, the catalyst is used in slight excess of equimolar quantities or more.

### DETAILED DESCRIPTION OF THE PREFERRED EMBODIMENT

The present process is applicable generally to the preparation of sulfonyl urea compounds of general Formula (I), and in particular the preparation of high purity sulfonyl urea drugs which are useful for therapeutic applications. Some of such medicinally useful compounds are drugs such as glimepiride, glipizide, gliclazide, glibenclamide, glibornuride, glisoxepide, etc. mentioned above.

In one aspect of the present invention, an efficient process for preparation of sulfonyl urea compound of Formula I 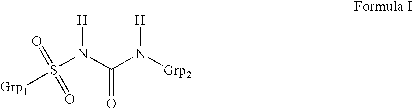
Wherein, Grp1 is a substituent group selected from residue A, B, C,D, E, or F given below 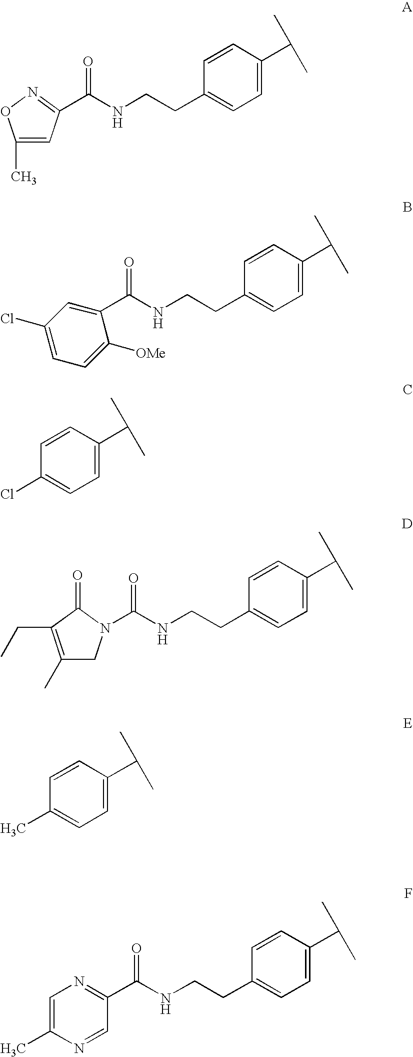
wherein Grp2 is a substituent selected from the residue P, Q, R, S, or T. 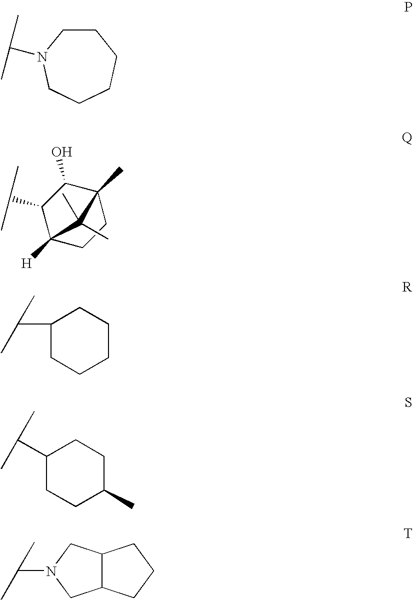
The point of attachment between Grp1 and Grp2 is derived from a reactive synthon, such as carbamate. Therefore, in one aspect of this invention, the process comprises the step of reacting a carbamate of general Formula IX with an amine compound of Formula X. The reaction is preferably carried out in the presence of an activation catalyst, preferably 4-pyrrolidinopyridine, 4-(dimethylamino)pyridine (DMAP), or the like, most preferably DMAP. The reaction may be efficiently carried out in an organic solvent medium, preferably a non-protic organic solvent, such as a hydrocarbon solvent. The organic solvent suitable for this conversion may be, but not limited to, aromatic hydrocarbon solvents, saturated hydrocarbon solvents, non-polar solvents, high boiling polar aprotic solvents, and combinations thereof.

The aromatic hydrocarbons may be, but not limited to, benzene, toluene, mono, di, or tri-substituted benzenes, xylene, and the like. High boiling polar solvents may be, but not limited to, dioxane, dimethyl formamide, dimethyl acetamide, dimethyl sulphoxide and the like. Preferably, the solvent is a high boiling aromatic hydrocarbon solvent, such as benzene, toluene, xylene, etc. The reaction is preferably carried out at reflux temperature with optional removal of the by-product (alcohol) from the reaction medium by distillation or any other means, such as Dean Stark arrangement.

The reaction is carried out by supplying heat, preferably at the boiling temperature of the solvent used. As the reaction proceeds, the by-product alcohol is accumulated in the reaction mass, which may be removed if volatile during the course of the reaction.

The activation catalyst is preferably used in molar amounts ranging from about 0.1 to 1.0 moles relative to the starting carbamate of Formula IX. The more preferred amount of catalyst is in the range of about 0.3 to 0.5 moles, and most preferably about 0.4 moles.

The amine compound of Formula X is preferably used in molar amounts or in excess ranging from about 1.0 to 1.5 relative to the carbamate (Formula IX) used, preferably about 1.0 to 1.3, and most preferably about 1.0 to 1.15.

The starting carbamate of Formula IX is prepared by known processes, such as reacting a sulphonamide of Formula XI with alkyl/aryl haloformate of formula XIII 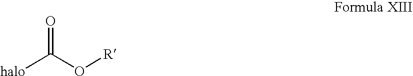
where halo is a halogen or pseudo halogen group; and R′ is an alkyl or aryl residue. In the process, preferably, the sulphonamide (Formula XI) is reacted with alkyl/aryl haloformate (Formula XIII) in presence of an organic base in a solvent selected from hydrocarbon and chlorinated hydrocarbon solvent. Chlorinated hydrocarbons may be, but not limited to, methylene chloride, ethylene chloride, chloroform, etc., with the preferred solvent being methylene chloride.

Preferably, the alkyl residue in the alkyl/aryl haloformate (Formula XIII) is a lower alkyl chain composed of lower alcohols. More preferably, it is ethyl group and the halogen is chlorine. This makes easy removal of the by-product in at a lower temperature by distillation.

The organic base may be, but not limited to, trialkyl amine, such as triethylamine and ethyldiisopropyl amine, 4-(dimethylamino)pyridine (DMAP), or the like. The preferred organic base is a combination of triethyl amine and DMAP.

The reaction is preferably carried out at a temperature ranging from about −10 to 35° C., and preferably, in methylene chloride. Preferably, the organic base may be used in a molar equivalent ratio relative to the sulphonamide of Formula XI.

On completion of reaction, the solvent is distilled and carbamate is isolated using a second solvent, such as acetone or toluene. The mass is then filtered and dried which gives a purity of at least 99.5%. However, it should be noted that the isolation method may vary from one sulfonyl urea compound to another depending upon the physico-chemical properties of each compound, which method may be distillation, extraction etc., that is apparent to a skilled artisan.

In an alternative embodiment of the present invention, a process is provided for preparation of sulfonyl urea compounds of Formula (I) by reacting compounds of Formula XI and Formula XII.

This process comprises reacting the compounds of Formula XI and Formula XII, preferably, in the presence of a base material and an activation catalyst, such as 4-pyrrolidinopyridine, 4-(dimethylamino)pyridine (DMAP), or the like. The reaction is efficiently carried out in presence of an organic solvent which may be selected from one of those mentioned earlier for the carbamate reaction. The process of this invention is characterized by higher conversion rate and faster reactions to yield the sulfonyl compounds (Formula I) in greater yield and purity.

The base material may be selected from organic or inorganic bases such as metal alkoxide, metal hydroxides, trialkyl amines etc. The metal alkoxides may be, but not limited to, sodium methoxide, sodium ethoxide, sodium/potassium tert-butoxide, etc. The metal alkoxide may be, but not limited to, sodium hydroxide, potassium hydroxide, sodium carbonates/potassium carbonates, etc. The trialkyl amine base may be, but not limited to, triethyl amine, disiopropylethyl amine, etc. Preferably, the base is a metal alkoxide, such as sodium methoxide.

In a preferred embodiment of the invention, the reaction is driven to completion by removal of the by-product, i.e., alcohol, from the reaction vessel continuously during the reaction. The means for effective removal of the alcohol formed in the course of reaction is preferably by distillation using a Dean-Stark arrangement or by normal distillation.

The sulfonyl urea compounds (I) obtained in the reaction are isolated in a conventional manner. In a typical procedure, the reaction mass after completion of the reaction is cooled to a temperature of −10 to 30° C., and simply filtered to obtain the crude sulfonyl urea compound (Formula I). In most of the cases, the purity of the crude product exceeds 99% (by HPLC area percent). The crude sulponyl urea compound (Formula I) may further be purified by precipitation from a suitable organic solvent, such as acetone, to obtain a pure sulfonyl urea end product in greater than 99.5% purity (HPLC assay). However, it should be noted that, the isolation method may vary from one sulfonyl urea compound to another depending upon the physico-chemical properties of each compound. The isolation method may be distillation, extraction, or the like, which is apparent to a skilled artisan.

The starting carbamate of Formula XII is prepared from the corresponding amino compound having the general formula NH₂-Grp2 (Formula X). The amino compound is reacted with a aryl/alkyl haloformate of Formula XIII in presence of a base material in the same manner as described for the carbamate of Formula IX.

The high purity pharmaceutically active sulfonyl urea compound of general Formula (I), obtained by the methods of the present invention, may be suitably incorporated in any conventional dosage form for administering to human patients. Apart from sulfonyl urea drug, such as glimepiride, gliclazide, etc, such pharmaceutical compositions may also contain other pharmaceutically acceptable additives and excipients. Conventional dosage forms include tablets, capsules, powders, injectibles, solutions, suspensions, etc.

The drastic yield and purity improvement produced by the present invention lead to an efficient and commercially acceptable synthetic process for the production of pharmaceutically valuable sulfonyl urea compounds.

Without further description, it is believed that one of ordinary skill in the art can, using the preceding description and the following illustrative examples, make and utilize the compounds of the present invention and practice the claimed methods. The following examples are given to illustrate the present invention. It should be understood that the invention is not to be limited to the specific conditions or details described in this examples.

### EXAMPLE 1

### Preparation of Carbamate of Formula IX (R=ethyl, and Grp1=D)

In a reaction vessel 100 g of 4[2-(3-ethyl-4-methyl-2-carbonyl pyrrolidine amido) ethyl] benzene sulfonamide, 72 g triethyl amine, and 1.6 L of dichloromethane were mixed and cooled to 0° C. 46.15 g of ethyl chloroformate was diluted separately with 200 mL of dichloromethane and added into the reaction vessel drop wise while maintaining the temperature at around 5° C. for 2 hours, and then at 25 to 30° C. until completion of the reaction. 2 L of water and 0.7 L of dichloromethane were added; and the pH of the reaction mass was adjusted to 4 by addition of acetic acid. The organic layer was then separated, washed with water, and concentrated to dryness. The residue was refluxed with 300 mL of acetone and cooled to 25 to 30° C., maintained for 1 hour, filtered, and washed with 100 mL of chilled acetone to obtain 100 g carbamate of Formula IX (yield 83%, purity 99.7%, and melting point: 177 to 182° C.).

### EXAMPLE 2

### Preparation of Glimepiride (Formula I, Grp1=D and Grp2=R)

In a reaction vessel, 80 g of carbamate of Formula IX, 27.2 g trans-4-methylcyclohexyl amine, 11.5 g 4-dimethylamino pyridine, and 1.6 L of toluene were mixed and heated to reflux. The toluene was distilled out, while maintaining total volume of the reaction constant. After completion of the reaction, the mass was cooled to 25 to 30° C. to precipitate the glimepiride which was filtered and washed with 800 mL of toluene. The filtered material was dried to get 88 gm (95% yield) of glimepiride (purity 99.5%).

80 g of glimepiride (obtained as above) was stirred with 800 mL of acetone at reflux temperature for 30 minutes, cooled to 25 to 30° C., filtered, and washed with 400 mL acetone. The filtered material was dried to produce 75 g glimepiride of 99.7% purity (by HPLC) with impurities of sulphonamide (Formula VI) and carbamate (Formula IX) at 0.2 and 0.05%, respectively. The melting point of the final product was 207 to 211° C.

Although certain presently preferred embodiments of the invention have been specifically described herein, it will be apparent to those skilled in the art to which the invention pertains that variations and modifications of the various embodiments shown and described herein may be made without departing from the spirit and scope of the invention. Accordingly, it is intended that the invention be limited only to the extent required by the appended claims and the applicable rules of law.

What is claimed is:

1. A process for making a compound of general formula (I), 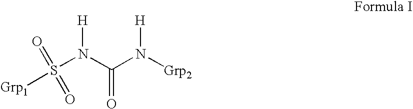
wherein Grp1 is any appropriately substituted or unsubstituted aryl residue and Grp2 is any (un)substituted cycloalkyl, polycyclic ring or heterocyclic ring, said process comprising either (a) reacting a combination of compound of formula IX and an amino-compound of Formula X, 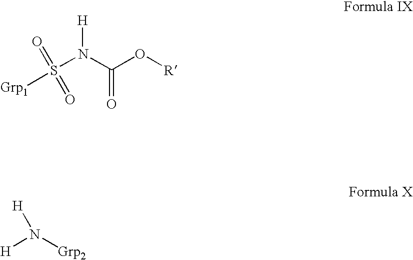
wherein R′ is an alkyl or aryl residue, in the presence of an activation catalyst; or (b) reacting a combination of a sulphonamide of Formula XI and a carbamate of Formula XII, 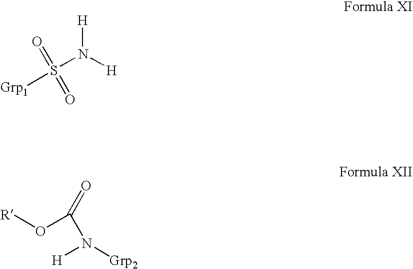
wherein R′ is an alkyl or aryl residue, in the presence of an activation catalyst.

2. The process as claimed in claim 1, wherein said reaction is performed in a solvent medium.

3. The process as claimed in claim 2, wherein said solvent medium is a organic solvent.

4. The process of claim 2, wherein said solvent medium is a hydrocarbon solvent, a non-polar solvent, or a polar aprotic solvent.

5. The process as claimed in claim 3, wherein said organic solvent is benzene, toluene, mono, di, or tri-substituted benzenes, xylene, dioxane, dimethyl formamide, dimethyl acetamide, dimethyl sulphoxide, or combinations thereof.

6. The process as claimed in claim 1, wherein said Grp1 is any one of the substituent group selected from residue A, B, C, D, E or F given below: 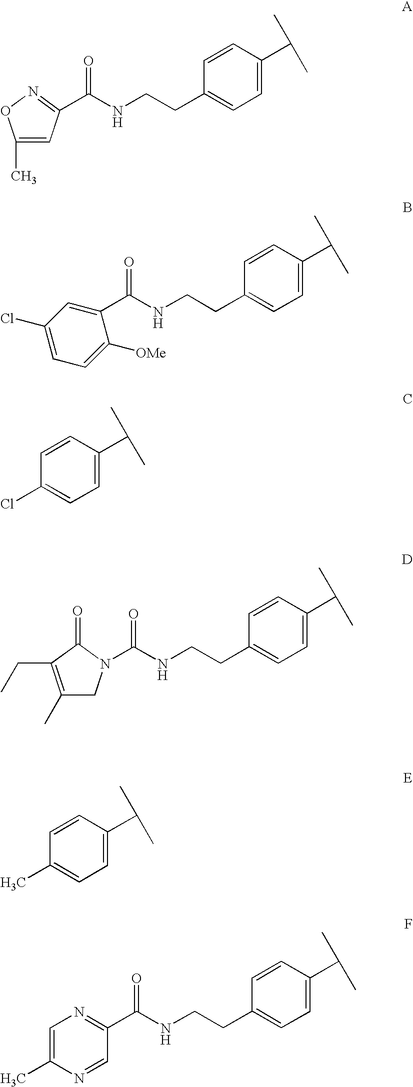
and said Grp2 is any one of the substituent group selected from residues P, Q, R, S, or T given below:

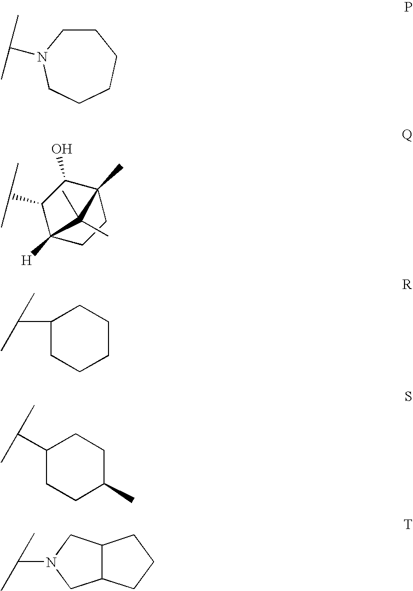

7. The process as claimed in claim 6, wherein a by-product alcohol is continuously removed from the reaction.

8. The process as claimed in claim 1, wherein said reaction of sulphonamide of Formula XI with carbamate of formula XII is in presence of a base.

9. The process of claim 8, wherein said base is an inorganic or organic.

10. The process as claimed in claim 8, wherein said base is alkali metal hydroxide, alklai metal carbonates or alkali metal alkoxides.

11. The process as claimed in claim 8, wherein said base is sodium methoxide or potassium carbonate.

12. The process as claimed in claim 1, wherein the compound (I) is glimepride, glipizide, glibenclamide, gliclazide, glibornuride, glisoxepide, and glypinamide.

13. The process as claimed in claim 1, wherein said starting carbamate of Formula IX is obtained by a process comprising:
i) combining corresponding sulphonamide of Formula XI with a haloformate compound of Formula XIII, 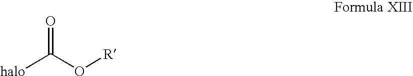
where in R′ is any alkyl or aryl residue, and halo is a halogen or pseudo halogen group; and
ii) reacting said combination in presence of an organic base in a solvent medium selected from a hydrocarbon solvent or a chlorinated hydrocarbon solvent.

14. The process of claim 19, wherein the organic base is a trialkyl amine.

15. The process of claim 20, wherein the trialkyl amine is triethylamine, ethyldiisopropyl amine, or a mixture thereof.

16. The process of claim 19, wherein said organic base is a combination of triethylamine and DMAP.

17. The process of claim 1, wherein the activation catalyst is 4-pyrrolidinopyridine or 4-(dimethylamino)pyridine (DMAP).
